(19) United States
(12) Patent Application Publication
(10) Pub. No.: US 2011/0054569 A1
(43) Pub. Date: Mar. 3, 2011

(54) PRESCRIPTION PAD FOR TREATMENT OF INFLAMMATORY DISORDERS

(76) Inventors: Ralph J. ZITNIK, Santa Barbara, CA (US); Michael A. Faltys, Valencia, CA (US)

(21) Appl. No.: 12/874,171

(22) Filed: Sep. 1, 2010

Related U.S. Application Data
(60) Provisional application No. 61/238,963, filed on Sep. 1, 2009.

(51) Int. Cl. A61N 1/08; A61N 1/36

(52) U.S. Cl. 607/59

(57) ABSTRACT

Described herein are devices, including interface modules or prescription pads, and systems including these devices and methods of using them, for treating inflammation or inflammatory disorders, and particularly for interfacing with a user desiring to prescribe treatment of an inflammatory disorder using an implanted stimulator.

### CROSS REFERENCE TO RELATED APPLICATIONS

This patent application claims priority to U.S. provisional patent application Ser. No. 61/238963, titled “PRESCRIPTION AND TREATMENT OF INFLAMMATORY DISORDERS,” filed on Sep. 1, 2009.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD OF THE INVENTION

Described herein are devices and systems for controlling one or more nerve stimulation devices for treating inflammatory disorders. In particular, described herein are interfaces and controllers for use with an implantable vagus nerve stimulation device for modulating the inflammatory reflex.

### BACKGROUND OF THE INVENTION

Inflammation is a complex biological response to pathogens, cell damage, and/or biological irritants. Inflammation may help an organism remove injurious stimuli, and initiate the healing process for the tissue, and is normally tightly regulated by the body. However, inappropriate or unchecked inflammation can also lead to a variety of disease states, including diseases such as hay fever, atherosclerosis, arthritis (rheumatoid, bursitis, gouty arthritis, polymyalgia rheumatic, etc.), asthma, autoimmune diseases, chronic inflammation, chronic prostatitis, glomerulonephritis, nephritis, inflammatory bowel diseases, pelvic inflammatory disease, reperfusion injury, transplant rejection, diabetes, vasculitis, myocarditis, colitis, etc. In autoimmune diseases, for example, the immune system inappropriately triggers an inflammatory response, causing damage to its own tissues.

The number of diseases characterized as inflammatory diseases or disorders is expanding almost daily.

The nervous system, and particularly the Vagus nerve, has been implicated as a modulator of inflammatory response. The Vagus nerve is part of an inflammatory reflex, which also includes the splenic nerve, the hepatic nerve and the trigeminal nerve. The efferent arm of the inflammatory reflex may be referred to as the cholinergic anti-inflammatory pathway. For example, Tracey et al., have previously reported that the nervous system regulates systemic inflammation through a Vagus nerve pathway. This pathway may involve the regulation of inflammatory cytokines and/or activation of leukocytes. Thus, it is believed that appropriate modulation of the Vagus nerve may help regulate inflammation.

Systems for stimulating one or more nerves of the cholinergic anti-inflammatory pathway (“CAP”) may include one or more electrical leads which may be implanted acutely or chronically, and may be positioned sufficiently near or in contact with the Vagus nerve or other nerves of the cholinergic anti-inflammatory pathway.

Application of electrical stimulation to a patient (e.g., via an implanted stimulation electrode(s), such as a vagal cuff electrode, or the like) to modulate inflammation may require control of various parameters (including pulse amplitude, duration, bursting (e.g., burst number, frequency, duration of burst), inter-pulse interval, intra-pulse interval, time-on and time-off, among others. Control of stimulation may also benefit from receiving and integrating feedback, including feedback based on markers (e.g., biomarkers), and patient/subject feedback. Control may also be based on client-specific parameters, including client response to various stimulation levels. These stimulation parameters may be tailored to specific ranges for treatment of one or more inflammatory disorders. Thus, it would be helpful to include devices and systems to help control the stimulation of a patient in a way that meaningfully integrates one or more of these parameters and allows useful interface with a physician or other medical practitioner. Described herein is one variation of such a system.

Also, described herein are various specific examples of disorders having an inflammatory component that may be treated by stimulation of the inflammatory reflex, including stimulation of the vagus nerve.

### SUMMARY OF THE INVENTION

The present invention relates treatment of inflammatory disorders, or disorders that implicate inflammation.

In particular, described herein are devices (e.g., interface modules), systems and methods for generating patient-specific neurostimulation parameters for treatment of inflammation or inflammatory disorders by stimulation of the inflammatory reflex. These devices, systems and methods may include an input for receiving patient-specific data, including patient identifying data (such as an implant identification code that identifies the patient based on their implanted stimulator). The same input may be used to enter a treatment prescription. In some variations the device or system proposes a treatment prescription based on the patient-specific data. The user (e.g., a physician) may then modify the proposed treatment prescription.

In some variations, when a user enters a treatment prescription the system may suggest modifications based on the subject-specific input.

A prescribed treatment may then be converted into a family of treatment regime parameters, which may be used to program the implanted stimulator. The treatment regime parameters may include, for example, the current or voltage intensity, a pulse width, a stimulation frequency, an on-time, and an off-time. The treatment regime may be based on both the prescription data as well as the patient-specific data; in some variations, the treatment regime may be modified based on feedback from the patient, including both self-reported feedback (e.g., patient-reported feedback) or feedback measured from the patient (biomarkers, physiological measurements, etc.)

In some variations the prescription pad may also monitor the status of the implanted stimulator, e.g., the charge, time to next charge, etc. The prescription pad may also be used to record or transmit treatment history or efficacy.

For example, described herein are interface modules for translating a prescribed treatment dosage for a patient into neuro stimulation parameters tailored to the patient for treatment of inflammation using an implanted neurostimulator to modulate the inflammatory reflex without substantially affecting the cardiac system, the interface module comprising. An interface module may comprise: an input configured to receive or modify a dosage input comprising a prescribed treatment dosage for the patient; a converter configured to convert the dosage input into patient treatment regime parameters comprising a current or voltage intensity, a pulse width, a stimulation frequency, an on-time, and an off-time; and an output configured to transmit the treatment regime parameters for receipt by an implanted neurostimulator.

In some variations, the patient treatment regime determined by interface module may include stimulation temporal characteristics configured to minimize the effects of the stimulation on the patient's cardiac system. For example, the voltage intensity, pulse width and timing (on/off durations) may be configured to be sub-threshold for modulating vagus nerve effects on heart rate. This may be empirically determined or determined specifically from each subject and provided as a patient-specific input to the interface module.

The interface module may be a prescription pad, and may be referred to as a prescription pad.

In general, the interface module may include hardware, software, firmware, or combinations and components thereof encompassing the input(s), converter(s) and output(s). Any appropriate hardware, software, firmware, or component may be used, as would be apparent to one of skill in the art under the direction of the description provided herein. For example, the converter may comprise a processor (e.g., a microprocessor) configured to receive the dosage input and to execute conversion logic to determine the patient treatment regime parameters. In some variations, the input of the interface module comprises a touch screen, a keypad, a mouse, a light pen, a tracking device, etc.

In any of the variations described herein, the converter may receive feedback information from the patient and to apply the feedback information to determine the patient treatment regime parameters. Feedback from the patient may be used to calculate or weight the determined treatment parameters.

In any of the variations described herein, the interface module may be configured to receive patient information that can be used by the converter (converter logic) to determine or constrain the patient treatment regime parameters. For example, patient-specific information (which may also include the feedback described above) may include patient age, weight, height, implant identification number, or the like. In addition, patient-specific information may include parameters of relevant clinical disease activity, including disease signs and symptoms, scores or score ranges for the patient which may signify disease remission or flare on one or more clinical assessment instruments. For example, scores on therapeutic tests such as the DAS (Disease Activity Score) in rheumatoid arthritis, or on the CDAI (Crohn's Disease Activity Index) in Crohn's Disease) may be provided to the interface module. Other patient-specific information that may be provided includes clinical laboratory values signifying disease remission or flare. For example the CRP (C-reactive protein) level, or other surrogate biomarkers from blood, biopsy or other tissue sources, may be provided, particularly those signifying disease remission or flare (or inflammation). For example, serum levels of pro-inflammatory cytokines, biopsy evidence of infiltration by disease-specific or non-specific types of inflammatory cells, and other derived indices of systemic inflammation including but not limited to changes in HRV (heart rate variability) may be provided.

The converter may propose a set of treatment regime parameters based on an input treatment dosage and on input patient-specific data and/or feedback. In some variations the converter may determine an initial set of treatment regime parameters based primarily on the patient-specific data and/or feedback, and allow a user to modify the treatment regime parameters. The presented treatment regime parameters may be displayed (or converted into and displayed) as a treatment dosage, and the treatment dosage may be modified by the user. For example, a user may input a treatment dosage comprising a prescription for the treatment of inflammation describing a specific energy (or energy range) over time, such as microcolumbs per minute, on a repeating schedule. As mentioned, an initial treatment dosage may be determined by the converter (from the patient-specific data) and presented to a user on the prescription pad for confirmation or modification. The treatment dosage may be converted into the treatment regime parameters that are later provided to the stimulator (or a programmer that programs the stimulator using the treatment regime parameters). In some variations the prescription pad displays or presenting the user with all or a subset of the treatment regime parameters.

In general, the converter may determine a set of treatment regime parameters using converter logic that may include one or more look-up tables, equations, or correlations, based on the provided input (including patient-specific and/or feedback data). In some variations, the converter establishes boundaries for the determined patient treatment regime parameters based by setting safety limits for the treatment regime parameters specific to the patient, and constraining the treatment regime parameters to be within the safety limits.

In general, the treatment regime parameters include parameters that may be used to program the stimulator and control its activity over the short or long term. For example, a set of treatment regime parameters may include a current or voltage intensity, a pulse width, a stimulation frequency, an on-time, and an off-time. The treatment regime parameters specified by the converter may also include a temporal pattern of stimulation, such as daily or weekly treatment times. In some variations, the treatment regime parameters may include increasing or decreasing stimulation intensity, power and/or frequency. For example, some of the treatment regime parameters (e.g., voltage/current, pulse width, frequency, on/off time, etc.) may be configured to change over the course of days, weeks, or months. For example, the treatment regime parameters may be configured to slowly increase stimulation level until the patient notices a decrease in symptoms, or (e.g., in patients whose disease is adequately controlled) to slowly decrease stimulation level while maintaining appropriate disease control in order to taper the therapy to minimum effective levels. In some variations, for example in patients experiencing therapy-related side effects, the treatment regime parameters may be configured to slowly decease the stimulation level to reduce such side effects while simultaneously maintaining appropriate disease control.

One variation described herein is an interface module for translating a prescribed treatment dosage for a patient into neurostimulation parameters tailored to the patient for treatment of inflammation using an implanted neurostimulator to modulate the inflammatory reflex. For example, an interface module may comprise: an input configured to receive or modify a dosage input comprising a prescribed treatment dosage for the patient, further configured to receive patient-specific data input; a converter configured to convert the dosage input and patient-specific data input into patient-specific patient treatment regime parameters comprising a current or voltage intensity, a pulse width, a stimulation frequency, an on-time, and an off-time; and an output configured to transmit the treatment regime parameters for receipt by an implanted neurostimulator.

Also described herein are systems for controlling the administration of electrical stimulation to treat inflammation from an implanted stimulator, the system comprising: an electrical prescription pad, the prescription pad comprising an input configured to receive or modify a prescribed treatment dosage, and a converter configured to convert the dosage input into treatment regime parameters comprising a current or voltage intensity, a pulse width, a stimulation frequency, an on-time, and an off-time; and an external controller configured to receive a treatment regime from the electrical prescription pad and to control an implanted controller.

As mentioned above, in some variations, the system further includes a charger configured to charge a battery in the implanted stimulator. The prescription pad may be configured to indicate the remaining change in the implant battery, and may provide an estimate or schedule for re-charging the implant. The charger may be coupled to the external controller, or it may be part of the external controller.

The prescription pad may also include an output configured to transmit the treatment regime to the external controller. An output may be a wireless output (including a transmitter and/or antenna, etc.) or a wired (connected) output.

Any of the variations of the interface module (e.g., prescription pad) described above may be included. For example, the dosage input may include a touch screen, a keypad, or a tracking device, etc. The prescription-pad may be configured to receive patient information, as described above, such as age, weight, height, implant identification number, etc. Furthermore, the converter may be configured to receive patient objective feedback information such as ECG, HRV, Blood Tests, including those from external tests or implanted sensors and recorders.

Also described herein are methods of prescribing the treatment of inflammation in a patient by stimulation of the inflammatory reflex from an implanted stimulator. These methods may include the steps of: entering a patient's identity into the electronic prescription pad; entering a patient's treatment prescription into an electronic prescription pad; converting the treatment prescription into a specific treatment regime for that patient, wherein the specific treatment regime comprises a current or voltage intensity, a pulse width, a stimulation frequency, an on-time, and an off-time; and transmitting the specific treatment regime to a controller configured to control operation of an electrical stimulator implanted in the patient.

The method may also include the step of entering feedback information from the patient into the prescription pad, wherein the feedback information is used to convert the treatment prescription into the specific treatment regime. The feedback may include any of the patient reported information (feedback) described above, including biomarker feedback, electrical nerve activity feedback or evoked compound action potentials, such as ECG, EMG, EEG, and diagnostic imaging techniques such as PET or fMRI. Nerve feedback may include peripheral vagal nerve evoked potentials, or a CNS readout such as an EEG brainstem or cortical evoked potential or functional MRI readout.

The method may also include the step of presenting a proposed treatment regime, allowing a user to modify the proposed treatment regime, and using the proposed treatment regime to determine the specific treatment regime. The step of entering the patient's treatment regime may include entering a dosage pattern with a specific energy in microcolumbs per minute on repeated schedule. The step of entering the patient's identity may include determining an identification number identifying the implanted stimulator that is correlated with patient identity. The method may also include entering the patient's diagnosis or medical history into the electronic prescription pad.

### BRIEF DESCRIPTION OF THE DRAWINGS

FIG. 1 is a schematic of one variation of a system for controlling the administration of electrical stimulation to treat inflammation from an implanted stimulator.

FIGS. 2A and 2B schematically illustrate two variation of another system for controlling the administration of electrical stimulation to treat inflammation from an implanted stimulator.

FIG. 3 illustrates one variation of an external controller and an interface module for an implanted stimulator.

FIG. 4 schematically illustrates one method of prescribing the treatment of inflammation in a patient by stimulation of the inflammatory reflex from an implanted stimulator.

### DETAILED DESCRIPTION OF THE INVENTION

Described herein are devices (including interface modules or prescription pads) and systems including the devices, as well as methods, for treating inflammation or inflammatory disorders, and particularly for interfacing with a user desiring to prescribe treatment of an inflammatory disorder using an implanted stimulator. Control and programming of a microstimulator to treat inflammatory disorders by stimulating the inflammatory reflex may be difficult. In particular, it may be difficult to coordinate all of the various stimulation parameters (such as voltage/current, on/off time, frequency, pulse width, and the like) to achieve an effective treatment that is specific or relatively specific to modulate the inflammatory reflex without undesirably effecting other systems, such as the cardiovascular system (e.g., heart rate).

In general, the devices and systems described herein may include an interface module, which may also be referred to (in some variations) as a prescription pad. The interface module may include one or more inputs for receiving or modifying a dosage input comprising a prescribed treatment dosage for the patient. The same input or a different input may be used to receive patient-specific data, including (but not limited to) feedback data. The device also typically includes a converter or converter module that is configured to convert a dosage input into a set of patient treatment regime parameters. The converter may also be configured to convert a set of patient treatment parameters into a dosage description. Patient treatment parameters typically include a current or voltage intensity, a pulse width, a stimulation frequency, an on-time, and an off-time. Other parameters particularly those regulating the programming and control of the stimulator, may be included, such as treatment time of day, daily/weekly/monthly treatment regimes (increasing or decreasing the “dosage” as desired over a particularly time period), or the like. In general, the devices and systems described herein also include one or more outputs configured to transmit the treatment regime parameters for receipt by an implanted neurostimulator.

The devices and system in this description may also be referred to as an “Immune System Regulators” (ISRs) because they may interrupt the inflammatory reflex, and thereby up- or down-regulate the immune system, particularly in response to disorder implicating inflammation. Examples of such disorders are provided below.

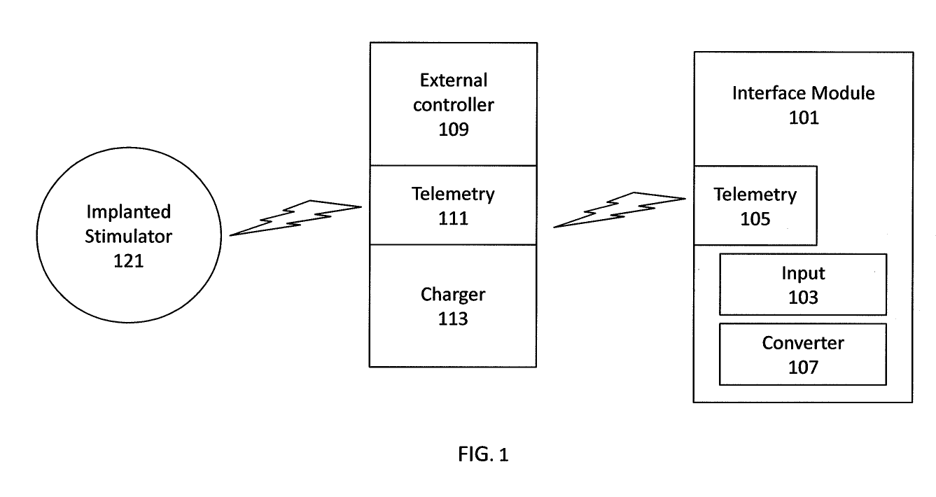

FIG. 1 shows a schematic illustrating one variation of an ISR that may be implemented with the implantable stimulator (e.g., a stimulating electrode array), where the IMPLANTABLE STIMULATOR is placed in the neck to stimulate the immune system. Any of the devices and systems described herein may be used with any appropriate stimulators, including implantable stimulators such as those illustrated herein.

In FIG. 1, a stimulator (e.g., an implanted stimulator) 121 has been previously implanted into a patient. The stimulator 121 receives programs/programming information from an external controller 109 via telemetry 111 connected to the external controller. In some variations a charger 113 is included. The charger 113 and (or) the telemetry 111 may be part of the external controller or they may be separate.

The stimulator may be programmed or controlled by a prescription pad (interface module 101) as illustrated. The prescription pad may also include telemetry 105 for communicating with the external controller and therefore the implant. As mentioned above, the interface module 101 typically includes one or more input(s) 103, and a converter 107. The input may be a manual input (e.g., keyboard, touch screen, mouse, etc.) or it may be a direct connection to one or more sensors. In some variations, the converter is hardware (e.g., circuitry), software, firmware, or some combination of the three. An output portion of the interface module 101 may be used to display the treatment parameters and/or dosage regimes.

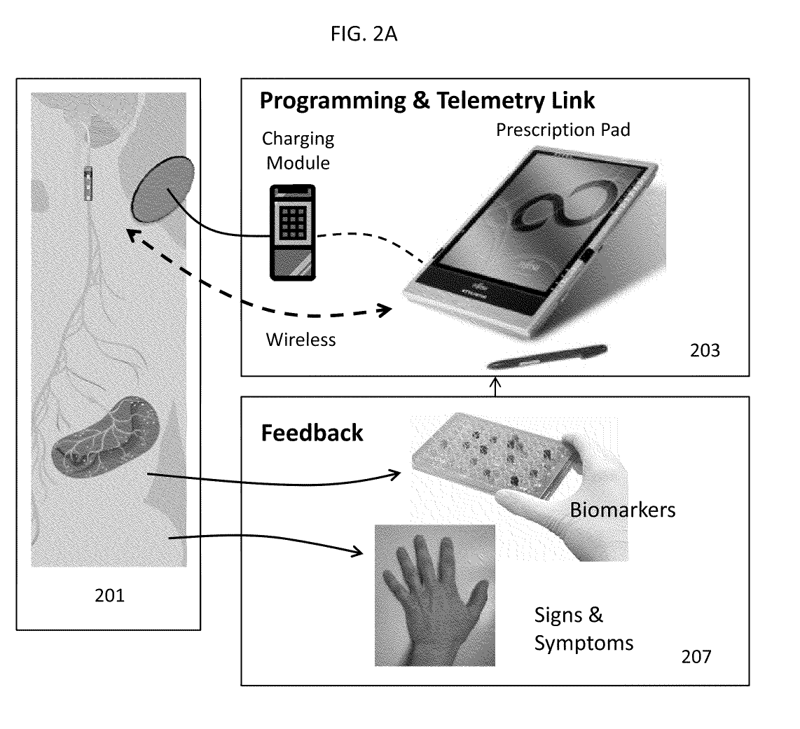

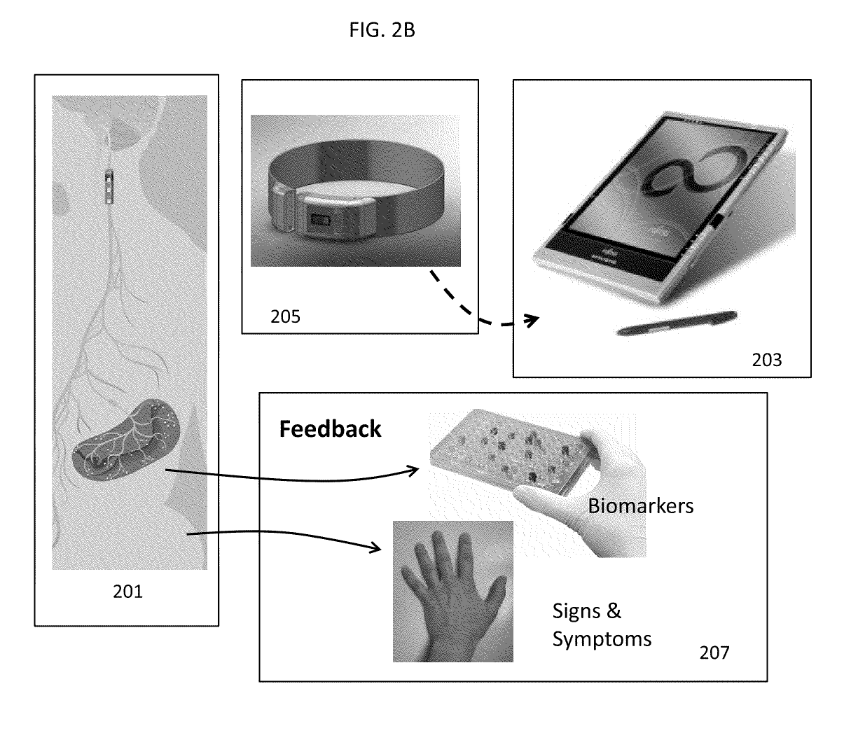

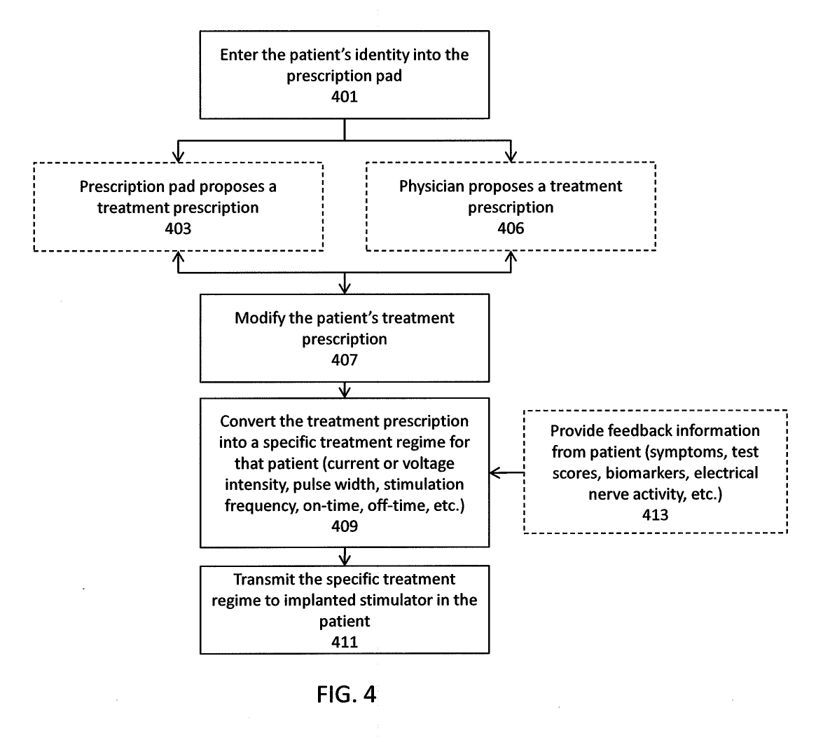

In operation, the device or system may operate as described in the flowchart of FIG. 4. Turning now to FIGS. 2A and 2B, schematic illustrations of the system are shown, highlighting the functions of the different regions. For example, in FIG. 2A the patient has had a stimulator (a microstimulator) implanted 201in electrical communication with the vagus nerve. An external programmer (including a “charging module”) is shown in communication with the implant, and with prescription pad 203. Patient feedback 207 may be fed back into the prescription pad, as indicated, and may help generate a set of treatment regime parameters or dosage. For example, a referring physician, e.g., a Rheumatologist, may send the patient to a specialist surgeon such as an ENT, or an interventionalist. The surgeon/physician may then place the prosthesis (IMPLANTABLE STIMULATOR) into the patient's neck next to the Vagus nerve, and uses the charging module to check the device.

Once the patient has healed from the implantation, the Rheumatologist can connect the prescription pad to the implanted device. The IMPLANTABLE STIMULATOR system example shown in FIG. 1A illustrates us two possibilities: one is a wireless link, the other a USB connection through the charging module. Other configurations are expressly contemplated.

A Rheumatologist may then work out a dose on their Prescription Pad, rather than rely on advanced knowledge of programming and electrical engineering to determine treatment, the Prescription may provide them simplified means for integrating a treatment plan into actual patterns of stimulation (e.g., a stimulation regime). When the Patient comes in to see the physician, the Rheumatologist can evaluate signs and symptoms such as joint swelling and optionally measure biomarkers. Rheumatologists currently bring in the patients every few months or weeks depending on their treatment. Applying this model will enable the Rheumatologist to regularly adjust the patient's prescription through the pad.

If the IMPLANTABLE STIMULATOR must be charged, this system is very low power consumption. Calculations show that the device could go 1-3 months between charges. This may allow the option to put charging in the hands of the clinician, rather than relying upon the patient.

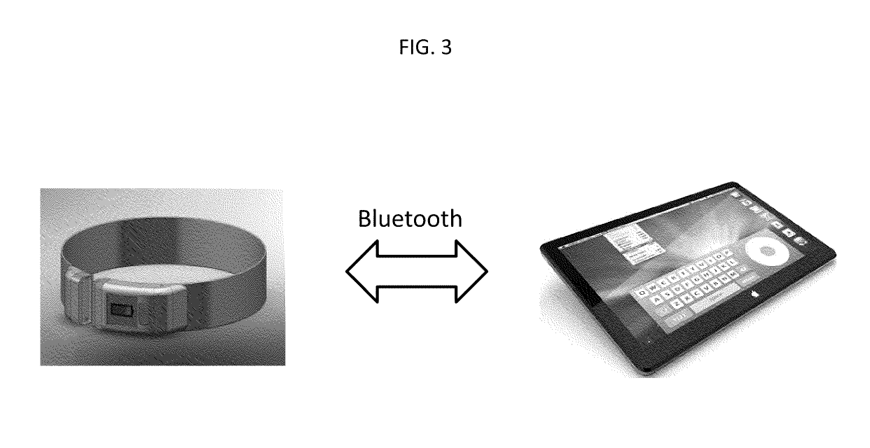

FIG. 2B illustrates another example of a system similar to that shown in FIG.1 except that the external controller element 205 is configured as a wearable device 205. In this example, the external controller is a necklace that may be worn to communicate with the implant during programming/charging. In use, a patient may be implanted with a stimulator 201, which, prior to use, has been checked and is ready to be programmed. The clinician or patient may then attach a programming necklace/charger 205, to the patient, turn on the prescription pad 203 and the prescription pad links to necklace over a network (e.g., Bluetooth network) This is exemplified in FIG. 3. The prescription pad may also receive input information (including patient identifying information) and feedback 207. For example, the implant may be keyed to include a unique identification code that identifies it, and this code may be read (or received) by the prescription pad. Other patient-specific data may be input into the prescription pad either directly, automatically and/or manually.

For example, in one variation once the patient has been implanted and an external controller has been activated, the clinician may enter patient information (e.g., name, age, sex, etc.) into the prescription pad. The prescription pad may then request to run a diagnostic and assign a unique id to patient based upon implant ID. The clinician then chooses a category of pathology, and the pad asks for additional information for the specific pathology relative to the patient. In this example, the pad assists the clinician in setting safety limits (maximum current and pulse width that is comfortable and safe for a patient). The pad may use any of this information to automatically generate a default program (prescription parameters). The user may then modify the generated default dosage. For example, the clinician may be queried on whether or not they would like to make changes.

In addition, the prescription pad may recommend care and maintenance to the patient, including a schedule for battery charging. Although the example shown in FIG. 2B above includes a pad configured with an external controller that is shown as a necklace charger, other variations are also contemplated. For example, in some variations the prescription pad and the external controller are integrated together, so that a separate external controller is not necessary.

During a follow-up visit, the patient may wear the external programmer (e.g., necklace) and the clinician may again downloads/input all the relevant information into the pad, such as feedback. The prescription pad may then advise the clinician of any problems and helps clinician solve them. As with the initial programming described above, the clinician may interview the patient and change the program (by changing all or a portion of the patient treatment regime parameters), or the pad may question the clinician on therapy efficacy and also ask the physician for objective test results such as CRP and cytokine assays. The pad may also automatically make recommendations for program changes.

As mentioned above, the pad may be connected to a network through a cell link, or other wireless means (or wired means). In some variations, the system may be configured so that, at any time, the physician may share their data with a central receiving/transmitting center (e.g., technical support). The prescription pad may also track patient history and can be backed up and restored from through the cell link.

FIG. 4 illustrates another variation of a method of prescribing the treatment of inflammation in a patient by stimulation of the inflammatory reflex from an implanted stimulator. In this example, the method may include the first step of entering the patient's identity into the prescription pad 401. This may be done automatically or manually, and allows the prescription pad to customize the treatment plan/dosage or programming to the individual wearing a particular (identified) implant. In some variations the prescription pad then receives information specific to the patient, such as physical characteristics of the patient (weight, age, height, gender, etc.) and the patient's condition (diagnosis, severity, biomarkers, etc.). The prescription pad may then (on this basis) generate a proposed treatment prescription (dosage) 403. Alternatively or in addition, the physician may propose and enter (input) a treatment prescription 406. Thereafter the physician may modify the patient's treatment prescription as desired 407. In some variations the prescription pad may continue to suggest refinements to the prescription (or modifications to the prescription) proposed by the physician. The dosage may be described in generic terms (e.g., power applied to the patient per unit time, duration of treatment, days of treatment, etc. The prescription pad may display to the physician the dosage and/or the treatment regime parameters (including the electrical/temporal properties), or a subset of these.

Once (or as) the dosage is determined, it may be converted from a “dosage” (e.g., microcolumbs per minute) into a specific treatment regime that is specific for that treatment 409. The patient specific data as well as the proposed treatment dosage and any received feedback 413 in particular, may be included in the conversion/determination of the set of treatment parameters. Thereafter, the specific treatment regime may be transmitted to program the stimulator implanted in the patient 411.

As mentioned above, any appropriate disease or disorder may be treated using appropriate stimulation of the inflammatory reflex (e.g., the vagus nerve). For example, the vagus nerve may be electrically (or mechanically) stimulated to treat any of the following diseases and disorders, including, but not limited to: Atherosclerosis; Myocardial infarction; Myocardial infarction (recurrence prophylaxis); Myocardial ischemia (angina); Takayasu's arteritis; Cardiomyopathy (dilated); Congestive heart failure; Deep venous thrombosis (prophylaxis); Endocarditis; Myocarditis; Pulmonary thromboembolism (prophylaxis); Aneurysm (inflammatory); Antiphospholipid syndrome; Arteritis; Cardiomyopathy (infiltrative); Henoch schoenlein purpura; Hypersensitivity (leukocytoclastic) vasculitis; Kawasaki's disease; Microscopic polyangiitis; Pericarditis; Polyarteritis nodosa (c-PAN); Pulmonary hypertension (cor pulmonale); Rheumatic fever; Temporal (giant cell) arteritis; Thromboangiitis obliterans; Thrombophlebitis; Any appropriate Dental, ENT (ear-nose-throat), and/or Ophthomalogical disorder, including, but not limited to:; Allergic rhinitis (Hay Fever); Ménière disease; Optic neuritis; Periodontal disease; Pharyngitis; Rhinitis; Sinusitis (acute); Sinusitis (chronic); Uveitis; Autoimmune inner ear disease; Glossitis; Oral lichen planus; Sympathetic ophthalmia; Any appropriate Dermatological disorder, including, but not limited to:; Psoriasis; Burns; Acne vulgaris; Angioedema; Atopic dermatitis (eczema); Discoid lupus; Neutrophilic dermatoses (Sweet's); Alopecia areata; Dermatitis; Dermatitis herpetiformis; Epidermolysis bullosa acquisita; Erythema nodosum; IgA linear dermatosis; Lichen planus; Lichen simplex chronicus; Pemphigoid (bullous); Pemphigus foliaceus; Pemphigus vulgaris; Pityriasis rosea; Pyoderma gangrenosum; Rosacea; Seborrheic dermatitis; Stevens-Johnson syndrome (EM); Sunburn; Urticaria (wheals); Vitiligo; Warts; Any appropriate Endo or Metabolic disorder, including, but not limited to:; Diabetes type II; Dysmetabolic syndrome (X); Glucose intolerance; Diabetes type I; Addison's disease; Cachexia; Goiter; Graves' disease; Hashimoto's thyroiditis; Hperpyrexia; Hypercholesterolemia; Hypertriglyceridemia; Thyroiditis; Toxic adenomas of the thyroid; Polyglandular autoimmunity type I; Polyglandular autoimmunity type II; Any appropriate blood and/or oncological disorder, including, but not limited to:; Graft-versus-host disease; Solid organ transplant rejection; Allograft (BMT) rejection (chronic); Felty's syndrome; Anemia of chronic disease (inflammation); Autoimmune hemolytic anemia; Autoimmune thrombocytopenic purpura; Hodgkin's disease; Pernicious anemia; Thrombotic thrombocytopenic purpura; Any appropriate GI disorder, including, but not limited to:; Crohn's disease; Hepatitis B virus; Hepatitis C virus; Nonalcoholic steatohepatitis (NASH); Pancreatitis (acute); Pancreatitis (chronic); Post-operative ileus; Ulcerative colitis; Celiac disease; Diverticulitis; Hepatitis; Acute colitis; Appendicitis; Autoimmune hepatitis; Cholangitis; Cholecystitis; Gastric ulcer; Irritable bowel syndrome; Ischemic colitis; Ogilvie's syndrome; Peptic ulcer disease; Peritonitis; Primary biliary cirrhosis; Primary sclerosing cholangitis; Pseudomembranous colitis; Achalasia; Duodenal ulcer; Enteritis; Epiglottitis; Granulomatous gastritis; Whipple's disease; Any appropriate ID disorder, including, but not limited to:; Sepsis (septicemia); Candidiasis; Dengue fever; Endotoxic shock; Lyme disease; Respiratory syncytial virus infection; Amebiasis; Creutzfeldt-Jakob disease; Filariasis; HIV infection; Hydatid cysts; Influenza; Malaria; Any appropriate MSK disorder, including, but not limited to:; Femoral head arthritis (osteoarthritis); Interphalangeal joint arthritis (osteoarthritis); Medial compartment arthritis (osteoarthritis); Osteoarthritis; Achilles tendonitis; Ankle sprain; Anterior cruciate ligament injury; Arthralgias; Arthritides; Bicipital tendonitis; Bursitis; Calcaneofibular ligament injury; Carpal tunnel syndrome; Cervical facet syndrome; Cervical spondylosis; Cubital tunnel syndrome; Flexor tenosynovitis; Gamekeeper's/Skier's thumb; Glenoid labrum injury; Groin pull; Hamstring injury; Hip pointer; Iliopsoas tendonitis; Iliotibial band syndrome; Infraspinatus syndrome; Jammed finger; Knee arthritis (osteoarthritis); Lateral collateral knee ligament injury; Lateral epicondylitis (tennis elbow); Little league elbow; Medial calf injury; Medial collateral knee ligament injury; Medial epicondylitis (golfer's elbow); Peroneal tendon subluxation; Peroneal tendon syndrome; Peroneal tendon tear; Peroneal tendonitis; Plantar fasciitis; Posterior cruciate ligament injury; Quadriceps contusion; Quadriceps partial muscle tear; Quadriceps strain (Jumper's knee); Retrocalcaneal bursitis; Rotator cuff impingement; Rotator cuff tendonitis; Spinal stenosis; Sportsman's hernia; Supraspinatus tendonitis; Swimmer's shoulder; Talofibular ligament injury; Tendonitis (acute); Tendonitis (chronic) AKA tendinosis; Trochanteric bursitis; Turf toe; Ulnar collateral ligament injury; Wrist arthritis; Wrist dislocation; Adductor tendonitis; Ankle impingement syndrome; Avascular necrosis; Calcium pyrophosphate deposition disease; Degenerative disk disease; Fasciitis; Glenohumeral arthritis; Gout; Herniated nucleus pulposis; Hip overuse syndrome; Lateral compartment arthritis; Lumbosacral facet syndrome; Medial synovial plica irritation; Meniscus tear; Metatarsalgia; Olecranon bursitis; Osteitis pubis; Osteochondritis dissecans; Osteomyelitis; Osteonecrosis (hip); Paget's disease; Palindromic rheumatism; Patellofemoral arthritis; Patellofemoral joint syndrome; Piriformis syndrome; Quadriceps tendon rupture; Quadriceps tendon sprain; Rotator cuff tear; Sacroiliac joint injury; Slipped capital femoral epiphysis; Spondylolisthesis (lumbrosacral); Synovitis; Tenosynovitis; Lumbar spondylosis; Pes anserine bursitis; Spondylolysis (lumbrosacral); Synovitis (transient); Any appropriate Neurological disorder, including, but not limited to:; Cerebral embolism/infarction (Stroke); Multiple sclerosis; Spinal cord injury; Traumatic brain injury (severe); Alzheimer's disease; Aseptic meningitis (including viral); Bell Palsy; Chronic inflammatory demyelinating polyradiculoneuropathy (CIDP); Encephalitis; Guillian-Barre syndrome; Headache; Meningitis; Migraine headaches; Paralysis; Acute disseminated encephalomyelitis; Marchiafava-Bignami syndrome; Myasthenia gravis; Myasthenic syndrome (Lambert-Eaton); Neuralgia (Trigeminal); Primary angiitis of the CNS; Stiff man syndrome; Tolosa-Hunt syndrome; Any appropriate OB-GYN disorder, including, but not limited to:; Dysmenorrhea; Endometriosis; Adenomyosis; Immune-mediated infertility; Infertility; Vaginitis; Cervical polyps; Preeclampsia; Septic abortion; Any appropriate Psychological disorder, including, but not limited to:; Obsessive compulsive disorder; Schizophrenia; Alcoholism; Depression; Any appropriate Renal or GU disorder, including, but not limited to:; Acute glomerulonephritis (post-strep); Berger's disease (IGA nephropathy); Chronic bacterial prostatitis; Epididymitis; Goodpasture's syndrome; Interstitial nephritis; Membranoproliferative glomerulonephritis (MPGN); Membranous nephropathy; Urethritis; Focal segmental glomerular sclerosis; Hemolytic Uremic Syndrome; Minimal change disease; Nephrotic syndrome; Renal artery stenosis; Renal Colic; Any appropriate Respiratory disorder, including, but not limited to:; Acute respiratory distress syndrome; Asthma; Chronic obstructive pulmonary disease; Idiopathic pulmonary fibrosis; Allergic bronchopulmonary aspergillosis; Alpha-1 antitrypsin deficiency; Bronchiolitis; Bronchiolitis obliterans organizing pneumonia; Bronchitis; Cystic fibrosis; Emphysema (centriacinar); Hypersensitivity pneumonitis (extrinsic allergic alveolitis); Pneumonitis; Radiation pneumonitis; Alveolitis; Bronchiectasis; Eosinophilic granuloma (histiocytosis X); Eosinophilic pneumonia (acute); Eosinophilic pneumonia (chronic); Idiopathic pulmonary hemosiderosis; Loffler's syndrome; Pleurisy (pleural effusion); Pneumoconiosis; Any appropriate Systemic disorder, including, but not limited to:; Systemic inflammatory response syndrome; Systemic lupus erythematosis; Ankylosing spondylitis; Complex regional pain syndrome (reflex sympathetic dystrophy); Allergy; Anaphylactic shock (anaphylaxis/environ. exposure); Behcet's syndrome; Dermatomyositis/Polymyositis; Fibromyalgia; Hemochromatosis; Hemorrhagic shock; Juvenile arthritis; Organ ischemia; Organ necrosis; Polymyalgia rheumatica; Relapsing polychondritis; Sarcoidosis; Sjogren syndrome; Amyloidosis (and subtypes); Churg-Strauss syndrome (allergic granulomatous angiitis); CREST syndrome; Essential mixed cryoglobulinemia; Familial Mediterranean fever; Immune complex disease (serum sickness); Inclusion body myositis; Lymphedema tarda; Mixed connective tissue disease; Reiter's syndrome (reactive arthritis); Scleroderma (systemic sclerosis); and Wegener's granulomatosis.

While the methods, devices and systems for programming a dosage of treatment into an implanted device for modulating the inflammatory reflex have been described in some detail here by way of illustration and example, such illustration and example is for purposes of clarity of understanding only. It will be readily apparent to those of ordinary skill in the art in light of the teachings herein that certain changes and modifications may be made thereto without departing from the spirit and scope of the invention.

We claim:

1. An interface module for translating a prescribed treatment dosage for a patient into neurostimulation parameters tailored to the patient for treatment of inflammation using an implanted neurostimulator to modulate the inflammatory reflex without substantially affecting the cardiac system, the interface module comprising:
an input configured to receive or modify a dosage input comprising a prescribed treatment dosage for the patient;
a converter configured to convert the dosage input into patient treatment regime parameters comprising a current or voltage intensity, a pulse width, a stimulation frequency, an on-time, and an off-time; and
an output configured to transmit the treatment regime parameters for receipt by an implanted neurostimulator.

2. The interface module of claim 1, wherein the patient treatment regime includes stimulation temporal characteristics configured to minimize the effects of the stimulation on the patient's cardiac system.

3. The interface module of claim 1, wherein the interface module is a prescription pad.

4. The interface module of claim 1, wherein the converter comprises a processor configured to receive the dosage input and to execute conversion logic to determine the patient treatment regime parameters.

5. The interface module of claim 1, wherein the converter is configured to receive feedback information from the patient and to apply the feedback information to determine the patient treatment regime parameters.

6. The interface module of claim 1, wherein the input comprises a touch screen, a keypad, or a tracking device.

7. The interface module of claim 1, wherein the input is further configured to receive patient information selected from the group consisting of: age; weight; height; implant identification number; parameters of relevant clinical disease activity including signs and symptoms; clinical assessment indicators of disease remission or flare; clinical laboratory values signifying disease remission or flare; surrogate biomarkers from blood, biopsy or other tissue sources; derived indices of systemic inflammation; and heart rate variability.

8. The interface module of claim 1, wherein the converter is configured to set safety limits for the treatment regime parameters specific to the patient, further wherein the treatment regime parameters are constrained to be within the safety limits.

9. The interface module of claim 1, wherein the treatment regime parameters specified by the converter comprise daily or weekly treatment times.

10. The interface module of claim 1, where the treatment regime parameters are configured to indicate that the parameters vary over the course of days, weeks, or months, increasing or decreasing stimulation level.

11. An interface module for translating a prescribed treatment dosage for a patient into neurostimulation parameters tailored to the patient for treatment of inflammation using an implanted neurostimulator to modulate the inflammatory reflex, the interface module comprising:
an input configured to receive or modify a dosage input comprising a prescribed treatment dosage for the patient, further configured to receive patient-specific data input;
a converter configured to convert the dosage input and patient-specific data input into patient-specific patient treatment regime parameters comprising a current or voltage intensity, a pulse width, a stimulation frequency, an on-time, and an off-time; and
an output configured to transmit the treatment regime parameters for receipt by an implanted neurostimulator.

12. A system for controlling the administration of electrical stimulation to treat inflammation from an implanted stimulator, the system comprising:
an electrical prescription pad, the prescription pad comprising
an input configured to receive or modify a prescribed treatment dosage;
a converter configured to convert the dosage input into treatment regime parameters comprising a current or voltage intensity, a pulse width, a stimulation frequency, an on-time, and an off-time; and
an external controller configured to receive a treatment regime from the electrical prescription pad and to control an implanted controller.

13. The system of claim 12, further comprising a charger configured to charge a battery in the implanted stimulator.

14. The system of claim 12, wherein the prescription pad further comprises an output configured to transmit the treatment regime to the external controller.

15. The system of claim 12, wherein the dosage input comprises a touch screen, a keypad, or a tracking device.

16. The system of claim 12, wherein the prescription-pad is configured to receive patient information selected from the group consisting of: age, weight, height, implant identification number.

17. The system of claim 12, wherein the converter is further configured to receive patient feedback information.

18. The system of claim 12, wherein the converter is configured to receive objective patient feedback information from external tests or implanted sensors and recorders.

19. The system of claim 12, wherein the converter is configured to set safety limits for the treatment regime parameters specific to a patient, further wherein the treatment regime parameters are constrained to be within the safety limits.

20. The system of claim 12, wherein the treatment regime parameters specified by the converter comprise daily treatment times.

21. The system of claim 12, wherein the prescription pad is further configured to indicate battery charging requirements for the implant.

22. A method of prescribing the treatment of inflammation in a patient by stimulation of the inflammatory reflex from an implanted stimulator, the method comprising:
entering a patient's identity into the electronic prescription pad;
entering a patient's treatment prescription into an electronic prescription pad;
converting the treatment prescription into a specific treatment regime for that patient, wherein the specific treatment regime comprises a current or voltage intensity, a pulse width, a stimulation frequency, an on-time, and an off-time; and
transmitting the specific treatment regime to a controller configured to control operation of an electrical stimulator implanted in the patient.

23. The method of claim 22, further comprising entering feedback information from the patient into the prescription pad, wherein the feedback information is used to convert the treatment prescription into the specific treatment regime.

24. The method of claim 23, wherein the feedback comprises patient reported feedback, biomarker feedback, electrical nerve activity feedback.

25. The method of claim 23, wherein the feedback consists of at least lone of: evoked compound action potentials; diagnostic imaging techniques; ECG; EMG; EEG; PET scan data; and fMRI data.

26. The method of claim 22, further comprising presenting a proposed treatment regime, allowing a user to modify the proposed treatment regime, and using the proposed treatment regime to determine the specific treatment regime.

27. The method of claim 22, wherein the step of entering the patient's treatment regime comprises entering a dosage pattern with a specific energy in microcolumbs per minute on repeated schedule.

28. The method of claim 22, wherein the step of entering the patient's identity comprises determining an identification number identifying the implanted stimulator that is correlated with patient identity.

29. The method of claim 22, further comprising entering the patient's diagnosis or medical history into the electronic prescription pad.
